(19) United States
(12) Patent Application Publication
(10) Pub. No.: US 2025/0204933 A1
(43) Pub. Date: Jun. 26, 2025

(54) METATARSOPHALANGEAL JOINT REVERSE LOAD BEARING IMPLANT

(71) Applicants: Michael RIVLIN, Moorestown, NJ (US); Ashkan Sedigh, Philadelphia, PA (US); Joseph N. Daniel, Philadelphia, PA (US)

(72) Inventors: Michael RIVLIN, Moorestown, NJ (US); Ashkan Sedigh, Philadelphia, PA (US); Joseph N. Daniel, Philadelphia, PA (US)

(21) Appl. No.: 19/002,173

(22) Filed: Dec. 26, 2024

Related U.S. Application Data
(60) Provisional application No. 63/614,789, filed on Dec. 26, 2023.

(51) Int. Cl. A61B 17/17; A61F 2/30; A61F 2/46

(52) U.S. Cl. CPC A61B 17/1775; A61F 2/30749; A61F 2/4606

(57) ABSTRACT

A foot metatarsal bone implant system including a press fit implant configured for stabilizing the foot metatarsal bone includes concave and convex components configured for mounting in the MTP joint. The implant system includes multiple implant sizes available to ensure proper fit for different patients. The concave and convex portions may be grit-blasted to enhance osteo-integration with the patient's bone. The metatarsal component may include a talon design for increased implant purchase and prevention of implant dislocation. The implant system may be configured for conversion to arthrodesis without removing the intramedullary components, facilitating disengagement of joint components.

### CROSS-REFERENCE TO RELATED APPLICATIONS

The present application claims the benefit of U.S. Provisional Patent Application No. 63/614,789, filed Dec. 26, 2023 and titled, “Metatarsophalangeal Joint Reverse Load Bearing Implant,” the entire contents of which is incorporated herein by reference in its entirety.

### BACKGROUND OF THE INVENTION

The present disclosure relates to the field of orthopedic implants and, more specifically, to a foot metatarsal bone implant system. The system is designed to provide increased stability and anatomical range of motion for patients suffering from metatarsophalangeal joint (“MTP”) arthritis of the great/big toe, offering a comprehensive solution for addressing foot first metatarsal bone pathology.

Great toe arthritis at the MTP joint (hallux rigidus) is a common condition that can lead to pain, impaired mobility, and potential complications. Various surgical approaches and implant systems have been developed to address the pathology but all fail to either preserve motion (arthrodesis options) or fail to provide a stable MTP artificial joint (arthroplasty options). Accordingly, there is a need for an improved implant system that offers enhanced stability, adaptability, and range of motion while minimizing bone resection during the implantation procedure.

### BRIEF SUMMARY OF THE INVENTION

The present disclosure relates to a foot metatarsophalangeal joint implant system designed to enhance stability and improve range of motion in patients suffering from great toe metatarsal bone arthritis. The preferred invention encompasses a novel approach to the MTP joint by moving the fulcrum proximal in the axis of the rotation and reversing the relationship between the head of the metatarsal (ball) and proximal phalanx (socket) increasing the patient's ability to weight bear and endure ground forces. The system includes various components and features that allow for a customizable implantation procedure while minimizing bone resection. The implants are available in different sizes and processed to promote osteo-integration with the patient's anatomy. Interlock fixation and modular design further enhance stability and range of the motion. The asymmetric liner/polyethylene component improves joint biomechanics, while the dorsal talon design prevents implant dislocation. Additionally, the system facilitates correction of valgus misalignment and offers conversion to arthrodesis without removing the intramedullary components. The described foot metatarsal bone implant system provides surgeons with a comprehensive solution for metatarsal bone instability while ensuring patient-specific anatomy and case of implantation.

In another aspect, the preferred invention is directed to a great toe metatarsophalangeal joint or foot metatarsal bone implant system including a press fit or cemented implant for stabilizing the foot metatarsal bone. The implant system may include an aspherical concave or ball (phalangeal) component and a concave or socket (metacarpal) component that is implanted in the metacarpophalangeal metatarsophalangeal joint, an optional phalangeal (“P1”) and Metatarsal interlock for enhanced stability, an optional modular design for customizable implant configurations and an asymmetric liner/polyethylene component for improved joint mechanics. A metatarsal component with a P1 and metacarpal anchor hole for suture tape or anchor attachment may be included in the implant system. The implant system may include a procedure requiring a dorsal approach to the hallux metatarsophalangeal joint without the need of lateral access (minimally invasive to preserve bone stock), cutting guides designed to minimize bone resection during the implantation procedure, a set comprising various broaches for implant preparation and multiple implant sizes available to ensure proper fit for different patients. The implant system may further include an optional patient specific design using three-dimensional (“3D”) mapping and analysis for MTP axis and bone structure via MRI/CT scan, grit-blasted or other processed surfaces on the implants to enhance osteo-integration with the patient's bone, a talon dorsal design on the metatarsal component for increased implant purchase and prevention of implant dislocation, a customizable spherical to aspherical design of the distal component based on stability requirements and patient factors, multiple polyethylene or other bearing insert options to promote a normal joint range of motion including asymmetric corrective options and a design configuration allowing for correction of valgus or varus misalignment and facilitating the use of tissue reconstruction using suture tape, suture, biologic ligament alternatives and biotenodesis screws for alignment enhancement. The implant system is preferably configured for ease of explanation using dedicated instrumentation included in the set that lock in or temporarily connect to the implant for extraction. The implant system may facilitate conversion to arthrodesis without removing the intramedullary components, facilitating disengagement of joint components. The concave and convex components may include constant or non-constant (aspherical) radii which can be different in each plane to facilitate differential congruence and to allow improved stability.

The implant system may be 3D manufactured for a cage like a hollow structured design for optimal ingrowth. The implant system may further include a screw hole for polyethylene fixation that also serves as an anchor for conversion to arthrodesis and has a variable angle locking feature that can go through the hollow phalangeal implant or through the phalanx explanting the phalangeal component.

### BRIEF DESCRIPTION OF THE SEVERAL VIEWS OF THE DRAWINGS

The following description of the disclosure will be better understood when read in conjunction with the appended drawings. It should be understood, however, that the disclosure is not limited to the precise arrangements and instrumentalities shown. In the drawings:

FIG. 1A illustrates a side elevational view of a preferred metatarsophalangeal joint (“MTP”) implant in accordance with a preferred embodiment of the present invention, wherein the implant preferably includes a polyethylene, metatarsal (socket) section or component and a phalangeal (ball or aspherical convex) section or component;

FIG. 1B illustrates a side perspective view of the preferred MTP implant of FIG. 1A;

FIG. 2 illustrates a rear perspective view of a concave proximal part or socket of a metatarsal section of the MTP implant of FIG. 1A;

FIG. 3 illustrates a side perspective view of a phalangeal (ball or aspherical convex) section or component of the MTP implant of FIG. 1A;

FIG. 4A illustrates a side perspective view of the MTP implant of FIG. 1A, in a post-fixation or mounted configuration in a foot of a patient, wherein the foot is partially transparent for clarity;

FIG. 4B illustrates an alternative side perspective view of the MTP implant of FIG. 1A, in the mounted configuration, wherein the foot is partially transparent for clarity;

FIG. 5 illustrates multiple side perspective views of various broaches for both the metatarsal and phalangeal stems of the MTP implant of FIG. 1A;

FIG. 6 illustrates a side perspective view of a fusion screw and fixation of the MTP joint in conversion to an arthrodesis of the MTP implant of FIG. 1A;

FIG. 7A illustrates a front elevational view of an alternative embodiment of an articulating portion geometry or aspherical convex section of of the phalangeal section of FIG. 3 of the MTP implant of FIG. 1A; and

FIG. 7B illustrates a side elevational view of the articulating portion geometry of the convex distal portion or component of FIG. 7A.

### DETAILED DESCRIPTION OF THE INVENTION

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the joint implant system, and designated parts thereof, in accordance with the present disclosure. In describing the joint implant system, the terms proximal and distal are used in relation to the heal of the patient, proximal being closer to the heal and distal being further from the heal. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

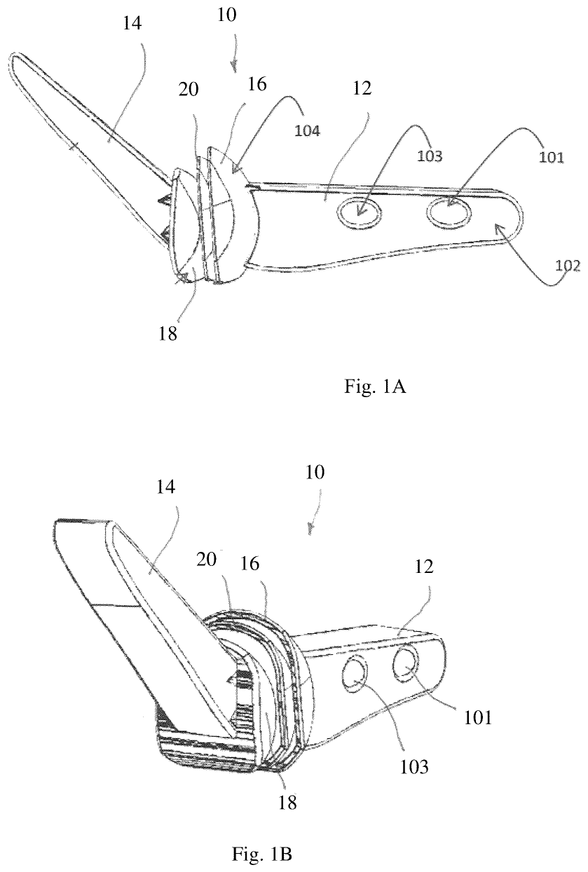

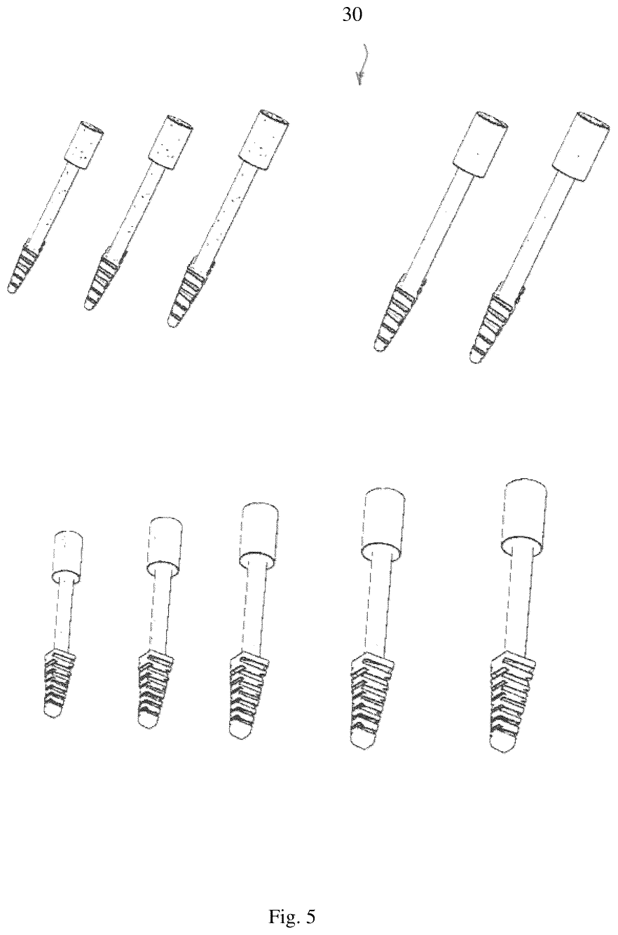

Referring to the drawings in detail, wherein like numerals indicate like elements throughout, there is shown in FIGS. 1-7B a first MTP joint implant system, generally designated 10, which overcomes the limitations of previous systems by providing a novel approach to stabilizing the foot metatarsal 401 while preserving joint functionality. The system 10 includes several components and features that contribute to its efficacy and versatility. The disclosure encompasses a novel approach to the MTP joint by moving the fulcrum proximal in the axis of rotation and reversing the relationship between the head of the metatarsal (convex joint or ball joint) and proximal phalanx (socket). This change of relationship creates a stable component (socket) on the metatarsal 401, which is the more robust and weightbearing bone compared to the proximal phalange 402. The design offsets the upward force on the MTP joint and rather than stressing the contact surface by the deforming shear forces, the stabilizing moment is now directed toward the metatarsal 401 in compression mode (minimizing shear). Furthermore, the design of the implant 10 is such that the socket is angled plantar, i.e., toward the sole of the foot, further using the upward force on the foot to create the reduction force on the implant 10 and lowering upward sheer on the implant system.

In one preferred embodiment, the implant 10 can be fixated through a press fit technique or using polymethacrylate or cement, providing surgeons with flexibility in choosing the preferred fixation method based on patient-specific needs. This ensures long-term implantation and promotes long-term stability by minimizing loosening of the implant 10 following surgery.

As shown, the joint implant system 10 is modular, having a metatarsal component 12 and a phalange component 14. Advantageously, the modularity of the system accommodates individual patient requirements, e.g., by allowing surgeons to customize the implant system 10 configuration based on a patient's specific condition, bone loss, and fixation needs, thereby providing optimal fit and stability. This design feature provides additional stability and ensures the long-term positioning of the implant 10. The metatarsal component 12 features a talon design, enhancing the implant's purchase, osteo-integration or connection to the metatarsal 401 when implanted and preventing dislocation, thereby providing additional stability and long-term fixation of the implant 10. The aspherical design of the articulating portion of the implant 10 allows precise biomechanics and boundaries in stability and range in various directions.

A distal end of the metatarsal component 12 takes the form of a concave proximal part or socket 16 and a proximal end of the phalange component 14 takes the form of a ball or aspherical convexity 18 configured to engage the concave proximal part or socket 16. Point of rotation is moved proximal and dorsal impingement is minimized by the aspherical design and repositions a center of rotation of the aspherical convexity 18 plantar and proximal. An asymmetric liner, e.g., a polyethylene component 20, improves joint mechanics and creates a roof for the upward ground reactive force further assisting stability. The unique design thereof promotes natural joint movement and increased range of motion, facilitating a more comfortable post-operative experience for the patient. The design allows for increased toe dorsiflexion as required with push-off and with shoes with an elevated heal or a healed shoe design. To prevent unforeseen dislocation of the components aspherical or asymmetrical design provides more shear strength to support stability. Moreover, this asymmetrical design provides an anatomical range of motion to the reconstructed joint. Moreover, the liner 20 assists with improving the joint stability by creating a compression force between the aspherical convexity or ball 18 and the concave proximal part or the socket 16. The liner 20 is preferably designed in multiple anatomic sizes to facilitate different patient anatomy. This can be custom fabricated based on initial templating, modelling or design based on imaging, 3D scan or other image acquisition and sizing techniques.

The joint implant system 10 may also include an interlock 103 that enhances implant stability. This interlock 103 provides additional resistance against implant dislocation, reinforcing the overall stability of the implant system 10 following surgery. For proper attachment and fixation, the metatarsal component 12 also includes metacarpal anchor or holes 101, 103, which allow for the attachment of suture or anchor for ligamentous reconstruction, further enhancing stability and providing additional support to the implant system 10. Metatarsal component 12 is not limited to including the anchor or holes 101, 103 and may alternatively include other features, such as spikes, treated surfaces or other bone connection features that facilitate fixation of the metatarsal component 12 to the metatarsal 401.

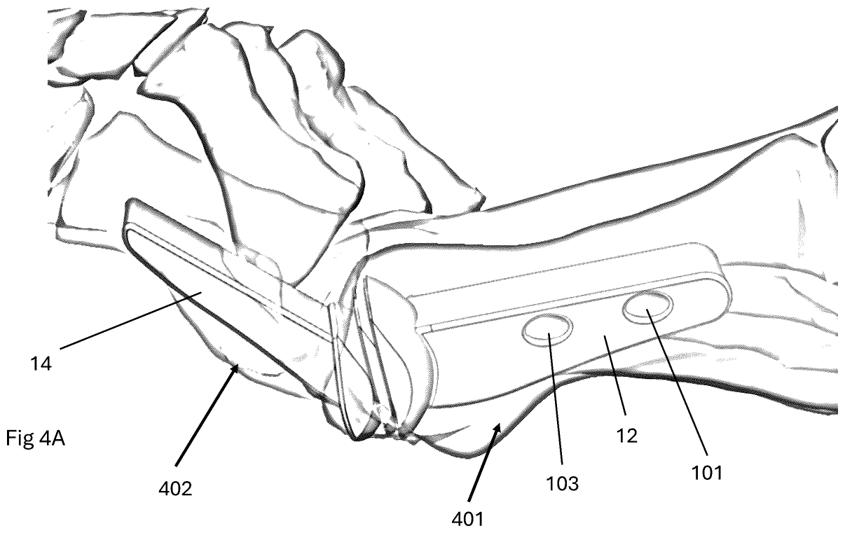

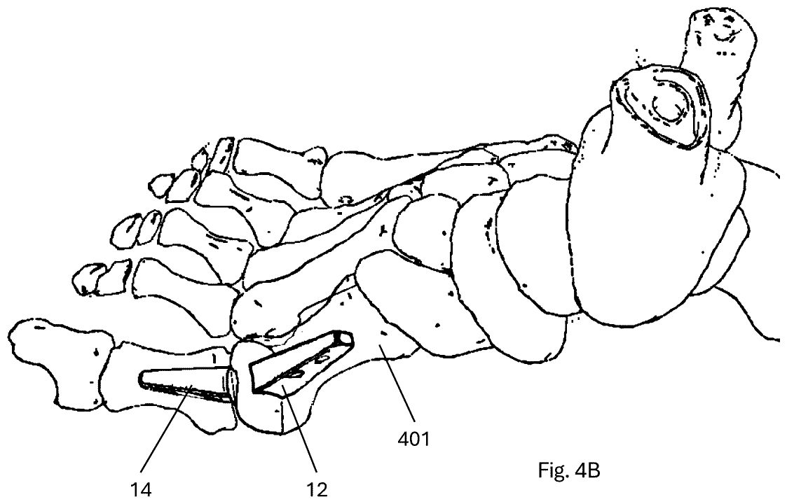

As shown in FIG. 4, a routine dorsal approach to the hallux metatarsophalangeal joint may be employed during the implantation procedure. This approach provides surgeons with a familiar and effective surgical technique for exposure of the metatarsophalangeal joint 401, facilitating the implantation process. To minimize bone resection, cutting guides 701 are specifically designed to optimize placement of the implant 10 while preserving as much native bone 401, 402, as possible. This minimally invasive approach reduces the risk of potential transfer metatarsalgia and enhances patient recovery. This technique maintains a weight distribution at the forefoot and thus minimizes the risk of potential transfer metatarsalgia and enhances normalization of gait.

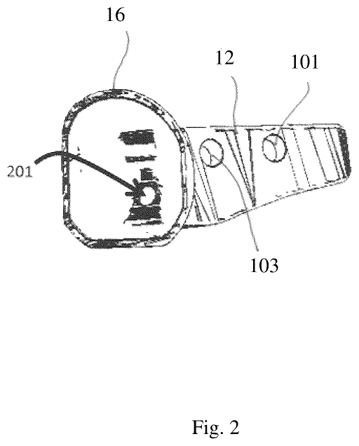

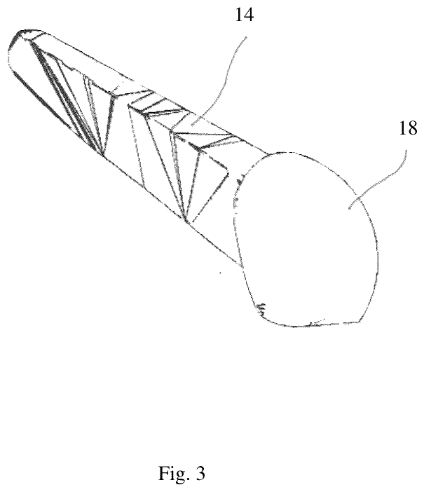

The foot metatarsal bone implant system 10 may be provided as a comprehensive set that includes a variety of differently sizes of broaches for implant preparation. Accordingly, a surgeon may select the appropriate broach size based on the patient's anatomy, to include deformity, ensuring accurate implant preparation. Trials are provided before final implant placement. The system 10 may also be constructed in different implant sizes (FIG. 2 and FIG. 3), ensuring a suitable fit for patients with varying metatarsal bone dimensions. This adaptability enables surgeons to address a wide range of patient needs and provide individualized treatment.

The surfaces of the implant system 10 may also be grit-blasted, or otherwise textured/processed, to enhance osteo-integration with the patient's bone. A textured surface promotes bone ingrowth, leading to a more secure and stable implant fixation. Additionally or alternatively, the implant 10 may be porous and 3D manufactured, yielding an integrated cage system for maximum ingrowth.

The system 10 is further configured to permit correction of valgus or varus misalignment. This correction is through asymmetric liner designs that can be trialed and selected. This feature allows surgeons to address misalignment during the implantation process. Offset (varus and valgus) and multiple sizes are used for the insert, which may be comprised of a polymeric insert. The offsets are graded in terms of valgus/varus angle and offer an asymmetric medial or lateral added material to enhance proper mechanical axis to minimize asymmetric wear and instability. The bearing components are preferably highly polished for optimal wear characteristics. Highly polished metal on polyethylene, or other bearing materials, such as ceramic bearing surfaces may be utilized.

The implant system 10 may be manufactured from materials that are themselves bearing materials (e.g., ceramics and the like), which do not require a polymeric bearing surface. The guide jigs allow for proper trialing of the implants 10 and predicts the need of ligamentous correction/repair or reconstruction. If such correction is required the system has built in facets or holes to facilitate anchor, suture or tape fixation and can accept bio-tenodesis screws for further alignment enhancement.

The implant system 10 may be manufactured for patient specific geometry using 3D image acquisition (Computer Tomography or Magnetic Resonance Imaging). Artificial Intelligence or manual implant design facilitates anatomic fit and predicts the need of bone substitution.

In the event removal of the implant 10 is required or desired, the great toe metatarsophalangeal joint or foot metatarsal bone implant system 10 includes dedicated explant instrumentation as part of the set. This ensures case of explantation and simplifies the revision procedure, if necessary.

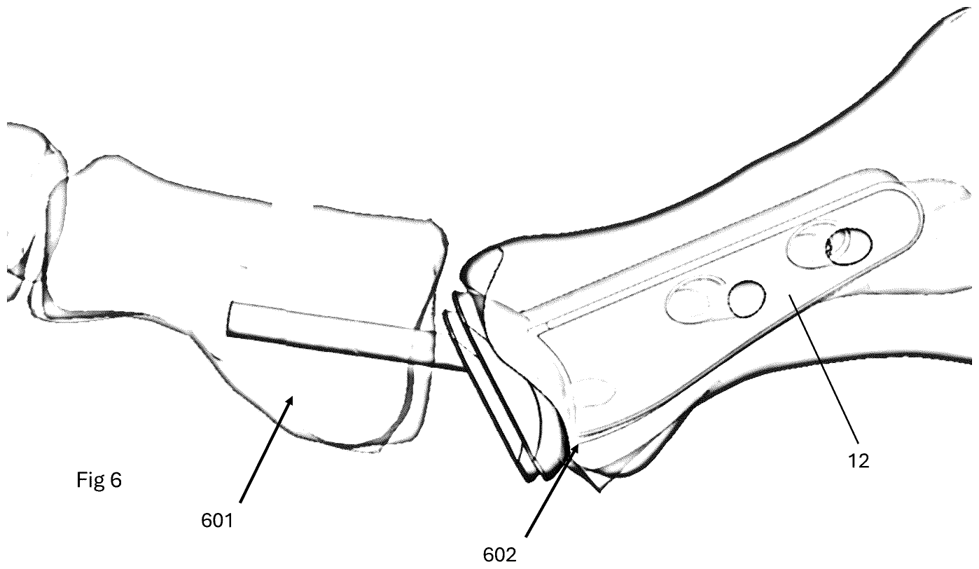

Moreover, the implant system 10 allows for conversion to arthrodesis without removing the intramedullary components. This feature provides surgeons with the ability to disengage the joint components and convert to arthrodesis, offering a valuable treatment option for specific cases. The arthrodesis screw can be either placed through the phalange component 14 or removing it into the metatarsal implant, which has a central channel for accepting the fusion device 601 (FIG. 6). The fusion screw 30 locks into a hole 201 on the metatarsal component 12 where the polyethylene fixation facet is located. The fusion screw hole 201 is designed to be multidirectional locking and the MTP axis angle can be selected prior to final tightening of the fusion screw 30, which converts the system to a full MTP fusion.

If the articulating MTP implant 10 fails, the phalange and metatarsal components 12, 14 may be connected and fixed together by placing the locking screw 30 that unites the two without any motion between them. In addition, the implant 10 may be modified by removing the phalangeal component 14 and screwing the phalanx with an intramedullary screw 601 into the metatarsal implant 12, thereby locking in the phalanx to the metatarsal component 12 so there is no motion between the metatarsal 401 and the phalange 402, thereby promoting fusion of the metatarsal 401 and the phalange 402.

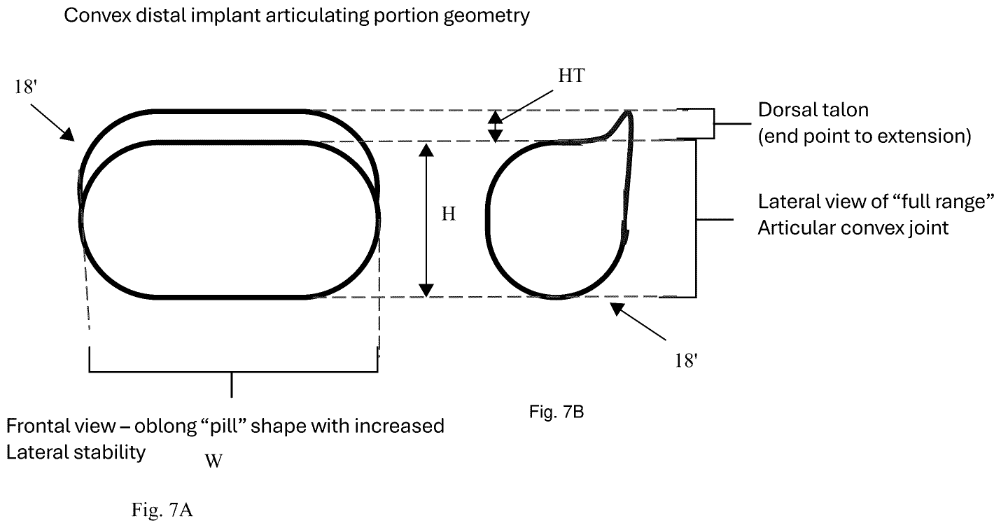

Referring to FIGS. 7A and 7B, the phalange component 14 may include an alternative aspherical convexity or ball section 18′ that mates with the concave proximal part or socket 16 of the metatarsal component 12. The alternative aspherical convexity or ball section 18′ has a similar configuration and function compared to the preferred aspherical convexity or ball section 18 of the preferred embodiment described above and is identified and distinguished from the preferred aspherical convexity or ball section 18 by a prime symbol (“′”). The alternative aspherical convexity or ball section 18′ includes a dorsal talon 19 extending dorsally from a distal section of the alternative aspherical convexity or ball section 18′. The dorsal talon 19 may contact a distal and dorsal section of the concave proximal part or socket 16 to limit the flexion of the patient's joint in the mounted configuration. The alternative aspherical convexity or ball section 18′ also preferably has an oblong width W that mates with a similarly oblong concave proximal part or socket (not shown) to improve lateral stability of the MTP joint implant 10 in the mounted configuration. The width W of the alternative aspherical convexity or ball section 18′ is greater than its height H and the width W and height H are both greater than a talon height HT of the dorsal talon 18 measured from a top of the alternative aspherical convexity or ball section 18′ and a dorsal tip of the talon 19.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the present description.

We claim:

1. A great toe metatarsophalangeal joint or foot metatarsal bone implant system comprising:
a press fit or cemented implant for stabilizing the foot metatarsal bone;
an aspherical concave or ball (phalangeal component) and concave or socket (metacarpal component) that is implanted in the metatarsophalangeal joint;
an optional phalangeal (P1) and Metatarsal interlock for enhanced stability;
an optional modular design for customizable implant configurations;
an asymmetric liner/polyethylene component for improved joint mechanics;
a metatarsal component with a P1 and metacarpal anchor hole for suture tape or anchor attachment;
a procedure requiring a routine dorsal approach to the hallux metatarsophalangeal joint without the need of lateral access (minimally invasive to preserve bone stock)
cutting guides designed to minimize bone resection during the implantation procedure;
a set comprising various broaches for implant preparation;
multiple implant sizes available to ensure proper fit for different patients;
optional patient specific design using 3D mapping and analysis for MTP axis and bone structure via MRI/CT scan
grit-blasted or other processed surface on the implants to enhance osteo-integration with the patient's bone;
a dorsal design on the metatarsal component for increased implant purchase and prevention of implant dislocation;
a customizable spherical to aspherical design of the distal component based on stability requirements and patient factors; —multiple polyethylene or other bearing insert options to promote a normal joint range of motion including asymmetric corrective options;
a design configuration allowing for correction of valgus or varus misalignment and facilitating the use of tissue reconstruction using suture tape, suture, biologic ligament alternatives and biotenodesis screws for alignment enhancement;
ease of explanation using dedicated instrumentation included in the set that lock in or temporarily connect to the implant for extraction; and
conversion to arthrodesis without removing the intramedullary components, facilitating disengagement of joint components, the concave and convex components include constant or non-constant (aspherical) radius which can be different in each plane to facilitate differential congruence and to allow improved stability.

2. The foot metatarsal bone implant system of claim 1, wherein the implant is fixated through a press fit technique.

3. The foot metatarsal bone implant system of claim 1, wherein the implant is fixated using cement.

4. The foot metatarsal bone implant system of claim 1, further comprising a P1 interlock integrated into the implant to provide additional stability.

5. The foot metatarsal bone implant system of claim 1, wherein the implant has a modular design allowing for different configurations based on patient anatomy.

6. The foot metatarsal bone implant system of claim 1, wherein the liner/polyethylene component is asymmetrically designed to improve joint mechanics.

7. The foot metatarsal bone implant system of claim 1, wherein the metatarsal component includes a P1 and metatarsal anchor hole for attachment of suture tape or anchor.

8. The foot metatarsal bone implant system of claim 1, wherein the implantation procedure involves a routine dorsal approach to the hallux metatarsophalangeal joint.

9. The foot metatarsal bone implant system of claim 1, wherein the cutting guides are designed to minimize bone resection during the implantation procedure.

10. The foot metatarsal bone implant system of claim 1, wherein the set comprises a variety of broaches for implant preparation.

11. The foot metatarsal bone implant system of claim 1, wherein multiple implant sizes are available to ensure an appropriate fit for different patients' anatomy.

12. The foot metatarsal bone implant system of claim 1, wherein the implants have a grit-blasted surface to enhance osteo-integration with the patient's bone.

13. The foot metatarsal bone implant system of claim 1, wherein the metatarsal component has a dorsal talon design to enhance implant purchase and prevent dislocation.

14. The foot metatarsal bone implant system of claim 1, wherein multiple polyethylene insert options are available to enhance a normal joint range of motion.

15. The foot metatarsal bone implant system of claim 1, wherein the design configuration allows for the correction of valgus misalignment and facilitates the use of suture tape, suture and tenodesis screws for alignment enhancement.

16. The foot metatarsal bone implant system of claim 1, wherein the implants can be easily removed using dedicated explant instrumentation included in the set.

17. The foot metatarsal bone implant system of claim 1, wherein the joint components can be disengaged to facilitate conversion to arthrodesis without removing the intramedullary components.

18. The foot metatarsal bone implant system of claim 1, further comprising an option for using PMMA (polymethyl methacrylate) for implant fixation.

19. The foot metatarsal bone implant system of claim 18, wherein the PMMA option provides additional stability and fixation support for the implant.

20. The foot metatarsal bone implant system of claim 1, wherein the metatarsal component and the phalangeal component are manufactured of a ceramic type material which doesn't require the addition of a polyethylene liner.
